(19) United States
(12) Patent Application Publication
(10) Pub. No.: US 2015/0282769 A1
(43) Pub. Date: Oct. 8, 2015

(54) IMAGE DISPLAY APPARATUS AND OPERATING METHOD THEREOF

(71) Applicant: SAMSUNG ELECTRONICS CO., LTD., Suwon-si (KR)

(72) Inventors: Ki-uk SONG, Seoul (KR); Duck-jin CHOI, Suwon-si (KR); Seok-myong KANG, Hwaseong-si (KR); Moo-rim KIM, Yongin-si (KR); Tae-gyu KIM, Hwaseong-si (KR)

(73) Assignee: SAMSUNG ELECTRONICS CO., LTD., Suwon-si (KR)

(21) Appl. No.: 14/681,654

(22) Filed: Apr. 8, 2015

(30) Foreign Application Priority Data
Apr. 8, 2014 (KR) 10-2014-0042020

(51) Int. Cl. A61B 5/00; A61B 5/145; A61B 5/11; A61B 5/021; A61B 5/0476

(52) U.S. Cl. CPC A61B 5/743; A61B 5/7282; A61B 5/021; A61B 5/0476; A61B 5/1118; A61B 5/14532; A61B 5/4806; A61B 2560/0242

(57) ABSTRACT

Provided are an image display apparatus and an operating method thereof. The operating method includes: periodically receiving, from an external device, health information of a user; matching and storing, in association with the user, the received health information; analyzing the stored health information; and outputting a health care service based on a result of the analyzing.

### CROSS-REFERENCE TO RELATED APPLICATION

This application claims priority from Korean Patent Application No. 10-2014-0042020, filed on Apr. 8, 2014 in the Korean Intellectual Property Office, the disclosure of which is incorporated herein in its entirety by reference.

### BACKGROUND

1. Field

Apparatuses and methods consistent with one or more exemplary embodiments relate to an image display apparatus and an operating method thereof, and more particularly, to an image display apparatus and an operating method thereof, in which health information of a user, received from an external device, is stored and a health care service is provided based on the stored health information.

2. Description of the Related Art

A recent rise in the standard of living has increased people's interest in health, and ubiquitous health care-related fields have been recognized as future growth engines along with the emergence of an aging society.

In line with this, technologies and industries associated with monitoring health have been actively studied, and techniques for collecting and managing measured health information by using various health sensors or devices have been variously developed. In particular, for a health care service, it is beneficial to collect and manage information on a real time basis without time or space limitations.

### SUMMARY

Aspects of one or more exemplary embodiments provide an image display apparatus and an operating method thereof, in which health information of a user is periodically collected from an external device and various health care services are provided based on the collected health information.

Additional aspects will be set forth in part in the description which follows and, in part, will be apparent from the description, or may be learned by practice of exemplary embodiments.

According to an aspect of an exemplary embodiment, there is provided an operating method of an image processing apparatus, the operating method including: periodically receiving, from an external device, health information of a user; matching and storing, in association with the user, the received health information; analyzing the stored health information; and outputting a health care service based on a result of the analyzing.

The receiving the health information and the storing the health information may be performed in a low-power mode of the image processing apparatus.

The receiving the health information and the storing the health information may be performed in the low power mode in which a main controller of the image processing apparatus is powered off.

The operating method may further include registering user information of the user, wherein the registered user information may include at least one of user Identification (ID) information and user authentication information.

The operating method may further include registering, in the image processing apparatus, the external device corresponding to the user, wherein the periodically receiving the health information may include periodically receiving the health information of the user from the registered external device.

The health information of the user may include at least one of biometric information, body weight information, body component information, brain wave information, blood pressure information, blood sugar level information, activity information indicating a user's activity, and sleep monitoring information of the user.

The operating method may further include receiving ambient environment information, wherein the analyzing the stored health information may include analyzing the stored health information of the user based on the received ambient environment information.

The ambient environment information may include at least one of a temperature, a humidity, a cleanliness level, and a Volatile Organic Compound (VOC) density.

The outputting the health care service may include outputting for display recommended content based on the analyzed health information.

The outputting the health care service may include outputting for display a health state of the user based on a result of the analyzing.

According to an aspect of another exemplary embodiment, there is provided an image display apparatus including: a health information monitor configured to receive and store health information of a user; a main controller configured to analyze the stored health information; and a display configured to display a result of the analyzing, wherein the health information monitor includes: a communicator configured to periodically receive, from an external device, the health information of the user; and a storage configured to match and store, in association with the user, the received health information.

The health information monitor may further include a sub controller configured to control the receiving and the storing of the health information, and the sub controller operates in a low power mode.

The sub controller may be configured to receive and store the health information even when the main controller is powered off.

The storage may be configured to store user information of the user, which may include at least one of user Identification (ID) information and user authentication information.

The storage may be configured to store external device information corresponding to the user, and the communicator may periodically receive the health information of the user from the external device corresponding to the stored external device information.

The health information of the user may include at least one of biometric information, body weight information, body component information, brain wave information, blood pressure information, blood sugar level information, activity information indicating a user's activity, and sleep monitoring information of the user.

The image display apparatus may further include a sensor configured to sense ambient environment information, wherein the main controller may be configured to analyze the stored health information of the user based on the received ambient environment information.

The ambient environment information may include at least one of a temperature, a humidity, a cleanliness level, and a Volatile Organic Compound (VOC) density.

The display may be configured to display recommended content based on the analyzed health information.

The display may be configured to output a health state of the user based on a result of the analyzing.

According to an aspect of another exemplary embodiment, there is provided a non-transitory computer-readable recording medium having recorded thereon a program for executing the method of the claim on a computer.

According to an aspect of another exemplary embodiment, there is provided an image processing apparatus including: a health information monitor configured to periodically receive health information of a user; and a main controller configured to analyze the received health information, wherein the health information monitor includes: a communicator configured to periodically receive, from an external device, the health information of the user; and a storage configured to match and store, in association with the user, the received health information.

The health information monitor may further include a sub controller configured to control the receiving and the storing of the health information in a low power mode.

The sub controller may be configured to receive and store the health information when the main controller is powered off.

The image processing apparatus may further include a main storage, wherein the main controller may be configured to copy or move the received health information, stored in the storage, to the main storage in response to the main controller being powered on, and wherein the main controller may be configured to analyze the health information stored in the main storage.

The storage may be configured to store user information of the user, which may include at least one of user Identification (ID) information and user authentication information.

The storage may be configured to store external device information corresponding to the user, and the communicator may periodically receive the health information of the user from the external device corresponding to the stored external device information.

The health information of the user may include at least one of biometric information, body weight information, body component information, brain wave information, blood pressure information, blood sugar level information, activity information indicating a user's activity, and sleep monitoring information of the user.

The image processing apparatus may further include a sensor configured to sense ambient environment information, wherein the main controller may be configured to analyze the stored health information of the user based on the received ambient environment information.

The ambient environment information may include at least one of a temperature, a humidity, a cleanliness level, and a Volatile Organic Compound (VOC) density.

The main controller may be configured to output recommended content based on the analyzed health information.

The main controller may be configured to output a health state of the user based on a result of the analyzing.

### BRIEF DESCRIPTION OF THE DRAWINGS

These and/or other aspects will become apparent and more readily appreciated from the following description of exemplary embodiments, taken in conjunction with the accompanying drawings in which:

FIG. 1 is a diagram for describing a health care system including an image display apparatus according to an exemplary embodiment;

FIG. 2 is a block diagram of an image display apparatus according to an exemplary embodiment;

FIG. 3 is a block diagram of a health information monitoring module according to an exemplary embodiment;

FIG. 4 is a flowchart of an operating method of an image display apparatus, according to an exemplary embodiment;

FIG. 5 is a flowchart of an operating method of an image display apparatus, according to an exemplary embodiment;

FIG. 6 illustrates a screen through which an external device is registered according to an exemplary embodiment;

FIG. 7 illustrates a screen on which a user's health state is displayed according to an exemplary embodiment; and

FIG. 8 illustrates a screen on which recommended content is displayed according to an exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, exemplary embodiments are merely described below, by referring to the figures, to explain aspects of the present description. Expressions such as “at least one of,” when preceding a list of elements, modify the entire list of elements and do not modify the individual elements of the list.

Terms used in the present disclosure have been selected as general terms which are widely used at present, in consideration of the functions of the exemplary embodiments, but may be altered according to the intent of an operator of ordinary skill in the art, conventional practice, or introduction of new technology. Also, if there is a term which is arbitrarily selected by the applicant in a specific case, a meaning of the term will be described in detail in a corresponding description portion of the exemplary embodiment. Therefore, the terms should be defined on the basis of the entire content of this specification instead of a simple name of each of the terms.

In the disclosure below, when the disclosure describes that a feature comprises (or includes or has) some elements, it should be understood that the feature may comprise (or include or has) only those elements, or the feature may comprise (or include or have) other elements as well as those elements, if there is no specific limitation. The term “unit” or “module”, as used herein, may refer to, but is not limited to, a unit for processing at least one function or operation, and may be implemented with hardware, software, or a combination thereof.

Hereinafter, exemplary embodiments will be described in detail with reference to the accompanying drawings to allow those of ordinary skill in the art to easily carry out the present disclosure. However, the present disclosure may be implemented in various ways, without being limited to exemplary embodiments described below.

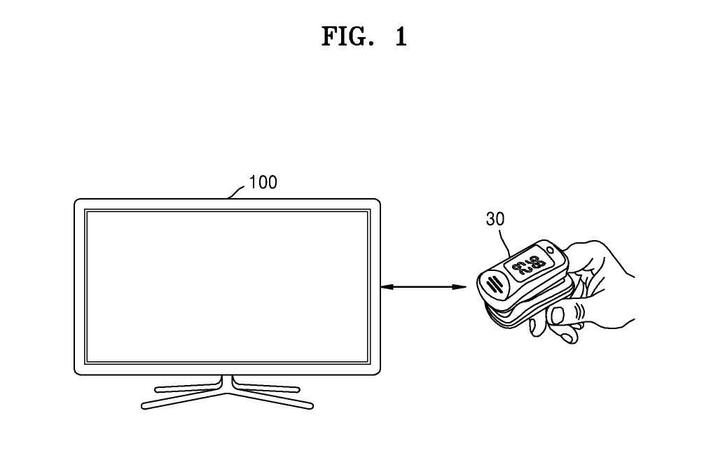

FIG. 1 is a diagram for describing a health care system including an image display apparatus 100 according to an exemplary embodiment.

Referring to FIG. 1, the health care system may include an image display apparatus 100 and at least one external device 30.

The image display apparatus 100 according to an exemplary embodiment may include various devices capable of processing and displaying an image signal. For example, the image display apparatus 100 may include a television (TV), a monitor, a cellular phone, a smart phone, a laptop computer, a tablet personal computer (PC), a digital broadcasting terminal, a Personal Digital Assistant (PDA), a Portable Multimedia Player (PMP), or the like. While the present exemplary embodiment is described with reference to an image display apparatus 100, it is understood that one or more other exemplary embodiments are not limited thereto, and may be applicable to an image processing device, such as a set-top box, an audio/video receiver, a media streaming device, etc., that outputs an image signal for display to an external display apparatus.

The external device 30 may include various devices capable of measuring health information. For example, the external device 30 may include a remote control device including a biometric information measurement sensor to measure biometric information, a mat or device capable of measuring a body weight or body components of a user, glasses capable of measuring brain waves of the user, a heartbeat measuring device for measuring a user's heartrate, a pulse measuring device for measuring a user's pulse, a tonometer for measuring a user's blood pressure, a blood sugar level measuring device for measuring a user's blood sugar level, a pedometer or calories measuring device for measuring a user's activity, a sleep monitoring device capable of measuring biometric information during sleep, a wearable smart device or fitness band, and so forth. It is understood that the above-described devices are examples of the external device 30 and any device capable of measuring data related to health information of a user may be the external device 30 without being limited to these examples.

The external device 30 transmits the measured health information to the image display apparatus 100 by using wired/wireless communication. The wired/wireless communication may include the Internet or short-range communication.

For example, the Internet technologies may include Wireless Local Area Network (WLAN), Wireless Fidelity (Wi-Fi), Wireless Broadband (WiBro), World Interoperability for Microwave Access (Wimax), High Speed Downlink Packet Access (HSDPA), and the like, and the short-range communication technologies may include Bluetooth®, Radio Frequency Identification (RFID), Infrared Data Association (IrDA), Ultra Wideband (UWB), ZigBee, and the like.

The image display apparatus 100 according to an exemplary embodiment periodically receives and stores health information (e.g., biometric information) from the external device 30, and analyzes a user state (e.g., a health state or a mood) based on the stored user health information to provide various health care services. In this regard, the image display apparatus 100 may periodically receive the health information according to predetermined intervals, a pre-set schedule, etc. Furthermore, the image display apparatus 100 may periodically receive the health information by periodically (e.g., according to predetermined intervals, a pre-set schedule, etc.) receiving, requesting, or pulling the information from one or more external devices (e.g., one or more pre-registered or detected external devices).

For example, the image display apparatus 100 may display at least one of the analyzed health state of the user and recommended content based on the analyzed health state or mood of the user. The image display apparatus 100 may also transmit the stored health information to an external server or an external device.

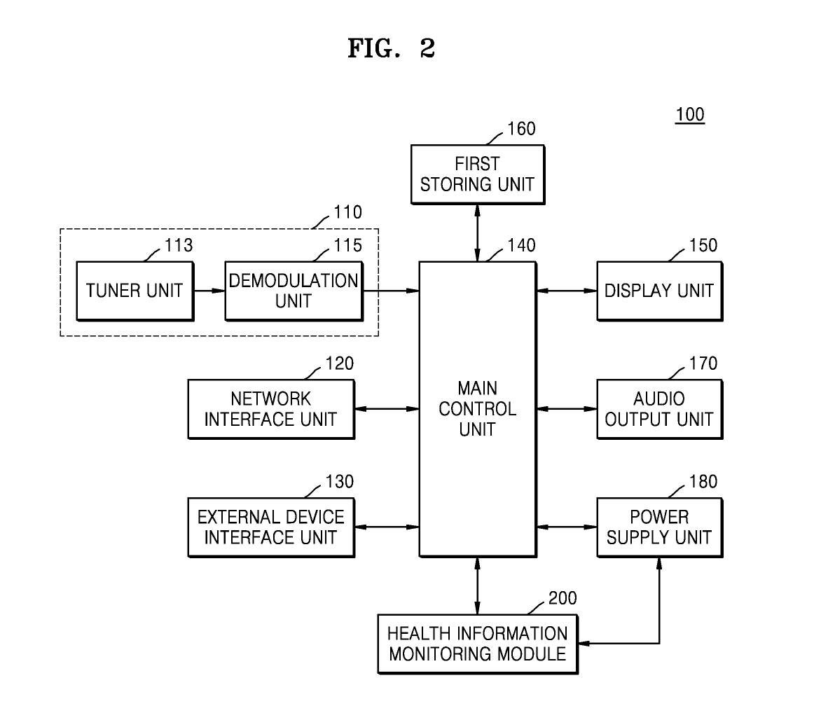

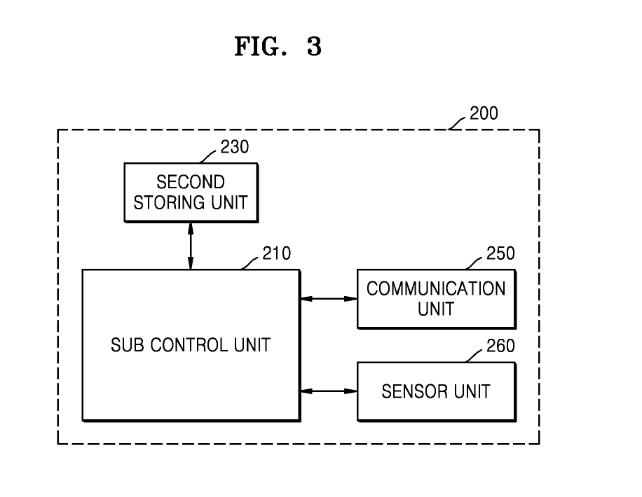

FIG. 2 is a block diagram of the image display apparatus 100 according to an exemplary embodiment, and FIG. 3 is a block diagram of a health information monitoring module 200 according to an exemplary embodiment.

Referring to FIG. 2, the image display apparatus 100 according to an exemplary embodiment may include a broadcasting reception unit 110 (e.g., broadcasting receiver), a main control unit 140 (e.g., main controller), a display unit 150 (e.g., display), the health information monitoring module 200 (e.g., health information monitor), an external device interface unit 130 (e.g., external device interface), a network interface unit 120 (e.g., network interface), a first storing unit 160 (e.g., first storage), an audio output unit 170 (e.g., audio outputter or audio output device), and a power supply unit 180 (e.g., power supplier).

The broadcasting reception unit 110 may include a tuner unit 113 (e.g., tuner) and a demodulation unit 115 (e.g., demodulator), although it is understood that one or more other exemplary embodiments are not limited thereto. For example, according to another exemplary embodiment, the broadcasting reception unit 110 may further include a network interface unit 120 (e.g., network interface) in addition to the tuner unit 113 and the demodulation unit 115. Alternatively, the broadcasting reception unit 110 may only include the network interface unit 120.

The tuner unit 113 may select a Radio Frequency (RF) broadcast signal corresponding to a user-selected channel or all previously-stored or configured channels from RF broadcast signals received via an antenna. The tuner unit 113 converts the selected RF broadcast signal into an Intermediate Frequency (IF) signal or a baseband image or voice signal.

For example, if the selected RF broadcast signal is a digital broadcast signal, the tuner unit 113 converts the selected RF broadcast signal into a Digital IF (DIF) signal, and if the selected RF broadcast signal is an analog broadcast signal, the tuner unit 113 converts the selected RF broadcast signal into an analog baseband image or voice signal (CVBS/SIF). That is, the tuner unit 113 may process a digital broadcast signal or an analog broadcast signal. The CVBS/SIF output from the tuner unit 113 may be directly input to the main control unit 140.

The tuner unit 113 may receive an RF broadcast signal of a single carrier according to the Advanced Television System Committee (ATSC) or an RF broadcast signal of multiple carriers according to Digital Video Broadcasting (DVB).

According to an exemplary embodiment, the tuner unit 113 may sequentially select RF broadcast signals of all broadcast channels stored by a channel memory function from among RF broadcast signals received via an antenna, and may convert the selected RF broadcast signals into IF signals or a baseband image or voice signals.

The tuner unit 113 may include a plurality of tuners to receive broadcast signals of a plurality of channels. The tuner unit 113 may also be a single tuner for simultaneously receiving broadcast signals of a plurality of channels.

The demodulation unit 115 receives a DIF signal from the tuner unit 113 and demodulates the DIF signal.

The demodulation unit 115 performs demodulation and channel decoding on the received DIF signal and outputs a stream signal that may be a signal in which at least one of an image signal, a voice signal, and a data signal is multiplexed.

The stream signal output from the demodulation unit 115 may be input to the main control unit 140. After performing demultiplexing and image/voice signal processing on the stream signal, the main control unit 140 outputs an image to the display unit 150 and outputs voice to the audio output unit 170.

The network interface unit 120 provides an interface for connecting the image display apparatus 100 with a wired/wireless network including, by way of example, an Internet network. For example, the network interface unit 120 may receive content or data provided by at least one of the Internet, a content provider, and a network operator through a network.

According to an exemplary embodiment, the network interface unit 120 may perform communication with the external device 30 so as to measure health information through a wired/wireless network. Thus, in this case, the image display apparatus 100 may receive user health information from the external device 30 or transmit the user health information through the network interface unit 120.

The external device interface unit 130 may transmit or receive data to or from an external device connected to the image display apparatus 100. To this end, the external device interface unit 130 may include a wireless communication unit (e.g., wireless communicator or wireless communication device) and an Audio/Video input and output unit (e.g., Audio/Video inputter and outputter or Audio/Video input and output device).

The external device interface unit 130 wiredly/wirelessly connects an external device such as a Digital Versatile Disk (DVD) player, a Blue-ray disk player, a game console, a camera, a camcorder, a computer (e.g., laptop), a set-top box, or the like to the image display apparatus 100 to transmit or receive data to or from the external device.

According to an exemplary embodiment, the image display apparatus 100 may receiver user health information from the external device 30 that measures health information or may transmit the user health information to the external device 30, through the external device interface unit 130.

The A/V input and output unit may receive an image and a voice signal of the external device. The wireless communication unit may perform short-range wireless communication with another electronic device. According to an exemplary embodiment, the wireless communication unit may perform short-range wireless communication (e.g., Bluetooth, Near Field Communication, Zigbee, local area wireless network, etc.) with the external device 30 that measures health information. Thus, the image display apparatus 100 may receive user health information from the external device 30 or transmit the user health information to the external device 30, through the wireless communication unit.

The image display apparatus 100 according to an exemplary embodiment may include the health information monitoring module 200 that will be described in detail with reference to FIG. 3.

Referring to FIG. 3, the health information monitoring module 200 may include a sub control unit 210 (e.g., sub controller), a second storing unit 230 (e.g., second storage), a communication unit 250 (e.g., communicator), and a sensor unit 260 (e.g., sensor).

The sub control unit 210 controls the health information monitoring module 200 to operate in a low-power mode. Thus, the sub control unit 210 controls the health information monitoring module 200 to operate with low power without being powered off even when the main control unit 140 is powered off.

The sub control unit 210 controls an overall operation of the health information monitoring module 200. For example, the sub control unit 210 may control a period for receiving health information from the external device 30, match the health information received from the external device 30 with user information, and store the matched health information in the second storing unit 230.

The communication unit 250 receives health information from the external device 30 capable of measuring the health information. The communication unit 250 may include at least one of an Internet module (e.g., network adapter) and a short-range communication module (e.g., short-range communicator).

The Internet module may receive health information from the external device 30 by using a wireless local area network (WLAN), Wi-Fi, wireless broadband (WiBro), WiMAX, High-Speed Downlink Packet Access (HSDPA), or the like.

The short-range communication module may receive health information from the external device 30 by using Bluetooth®, radio frequency identification (RFID), Infrared Data Association (IrDA), ultra-wideband (UWB), ZigBee, near field communication (NFC), or the like.

The health information received from the external device 30 may include a user's biometric information (for example, Electrocardiography (ECG) information, Photoplethysmography (PPG) information, Galvanic Skin Reflex (GSR) information, or the like), body weight information, body component information, brain wave information, blood pressure information, blood sugar level information, activity information indicating a user's activity, sleep monitoring information, or the like.

The second storing unit 230 stores user information for monitoring health information. The user information may include user identification information or authentication information. For example, the user information may include at least one of a user identification (ID), a password, user biometric information (for example, a user's fingerprint, a user's ECG pattern, a PPG pattern, a shape of a blood vessel of the back of the hand or a wrist of the user, a user's iris pattern, a user's voice, a user's face, and so forth), etc.

The second storing unit 230 matches and stores the received health information with stored user information. The sub control unit 210 recognizes which user among users registered in the image display apparatus 100 the received health information corresponds to, and matches and stores the received health information with its corresponding user information.

For example, the sub control unit 210 determines which user, among the users registered in the image display apparatus 100, the external device 30 having transmitted the health information is registered with, and matches and stores the received health information with its corresponding user information.

The sub control unit 210 compares the received health information with previously-stored user health information. Furthermore, if the health information patterns of the received health information and the previously-stored user health information coincide within a predetermined range, the sub control unit 210 matches and stores the received health information with its corresponding user information.

When receiving the health information, the sub control unit 210 may also receive user identification information or authentication information to compare the received user identification information or authentication information with previously-stored user identification information or authentication information. If the received identification information or authentication information coincides with the previously-stored user identification information or authentication information, the sub control unit 210 matches and stores the received health information with user information corresponding to the user identification information or authentication information.

The sensor unit 260 measures ambient environment information. For example, the sensor unit 260 may periodically measure temperature information, humidity information, cleanliness level information, Volatile Organic Compound (VOC) density information, and air pollution level information around the image display apparatus 100, and the measured ambient environment information may be stored in the second storing unit 230.

Referring back to FIG. 2, the main control unit 140, the first storing unit 160, the display unit 150, and the power supply unit 180 will now be described.

The main control unit 140 demultiplexes a stream input through the broadcasting reception unit 110 or processes demultiplexed signals to generate and output a signal for outputting an image or a voice. The image signal processed in the main control unit 140 may be input to the display unit 150 and may be displayed as an image corresponding to the image signal. The image signal processed in the main control unit 140 may also be input to an external output device through the external device interface unit 130.

The voice signal processed in the main control unit 140 may be output through the audio output unit 170. The voice signal processed in the main control unit 140 may also be input to the external output device through the external device interface unit 130. The main control unit 140 may control an overall operation of the image display apparatus 100.

According to an exemplary embodiment, the main control unit 140 analyzes a user's health state or mood based on the health information stored in the health information monitoring module 200.

For example, if the user turns on the image display apparatus 100 (for example, turns on the main control unit 140) or requests analysis of the user's health state, the main control unit 140 may analyze the user health state or mood based on the user health information stored in the health information monitoring module 200.

The main control unit 140 analyzes the user's health state or mood by using the ambient environment information and the health information, and analyzes recommended environment information based on the ambient environment information and the user health information. The recommended environment information may include recommended temperature, recommended humidity, and recommended cleanliness level of the ambient environment, depending on the user's health state.

The first storing unit 160 stores programs for signal processing and control in the main control unit 140 or a signal-processed image, voice, or data signal.

The first storing unit 160 performs a function for temporarily storing an image, voice, or data signal input to the external device interface unit 130. The first storing unit 160 stores information about a predetermined broadcasting channel through a channel memory function, such as a channel map.

According to an exemplary embodiment, the first storing unit 160 stores the health information and the ambient environment information stored in the second storing unit 230 of FIG. 3. For example, if the main control unit 140 of the image display apparatus 100 is turned on, the main control unit 140 stores (e.g., copies or moves) the health information and the ambient environment information stored in the second storing unit 230 in the first storing unit 160.

According to an exemplary embodiment, each time when the main control unit 140 is turned on, the first storing unit 160 accumulatively stores the health information and the ambient environment information stored in the second storing unit 230.

While FIG. 2 illustrates an exemplary embodiment in which the first storing unit 160 is provided separately from the main control unit 140, it is understood that one or more other exemplary embodiments are not limited thereto. For example, according to another exemplary embodiment, the first storing unit 160 may be included in the main control unit 140.

The main control unit 140 controls the display unit 150 to display a result of analysis with respect to the health information.

The display unit 150 converts at least one of an image signal, a data signal, an On-Screen Display (OSD) signal, and a control signal processed in the main control unit 140 or at least one of an image signal, a data signal, and a control signal received through the external device interface unit 130 to generate a drive signal. The display unit 150 may be a Plasma Display Panel (PDP), a Liquid Crystal Display (LCD), an Organic Light-Emitting Diode (OLED) display, a flexible display, a three-dimensional (3D) display, etc. The display unit 150 may be configured with a touch screen for use as an input device as well as an output device.

According to an exemplary embodiment, the display unit 150 displays recommended content based on the analyzed health state or mood of the user. If the user's health state is determined to need medical treatment, the display unit 150 may display a medical subject or a location of a hospital or drug store, together with a message indicating a need for medical treatment.

The audio output unit 170 outputs the voice signal processed in the main control unit 140. According to an exemplary embodiment, the audio output unit 170 outputs the voice signal processed in the main control unit 140 based on the analyzed health state or mood of the user.

The power supply unit 180 supplies power for operations of components with applied external or internal power. The power supply unit 180 may separately supply power to the health information monitoring module 200 to allow the health information monitoring module 200 to operate even when the main control unit 140 of the image display apparatus 100 is turned off.

Thus, even when the image display apparatus 100 is turned off, the health information monitoring module 200 may periodically receive and store user health information.

The block diagrams of the image display apparatus 100 and the health information monitoring module 200 illustrated in FIGS. 2 and 3 are block diagrams according to an exemplary embodiment. Each component of the block diagrams may be integrated, added, or omitted depending on specifications of the image display apparatus 100 and the health information monitoring module 200 that are actually implemented. That is, if necessary, two or more components may be integrated into one component or one component may be divided into two or more components. A function performed in each block is intended for description of an exemplary embodiment, and it is understood that one or more other exemplary embodiments may not be limited thereto.

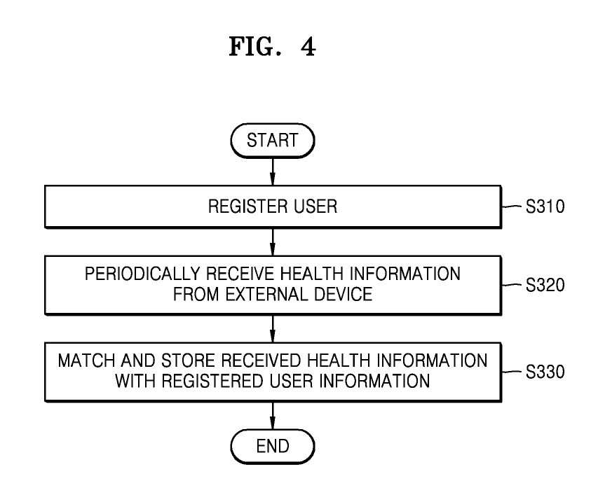

FIG. 4 is a flowchart illustrating an operating method of the image display apparatus 100, according to an exemplary embodiment.

Referring to FIG. 4, the image display apparatus 100 according to an exemplary embodiment registers a user for which health information is to be monitored and stores user information in operation S310. The user information may include user identification information or authentication. For example, the image display apparatus 100 may register at least one of a user ID, a password, user biometric information (for example, a user's fingerprint, a user's ECG pattern, a PPG pattern, a shape of the back of the hand or a wrist of the user, a user's iris pattern, a user's voice, a user's face, and so forth).

For example, the image display apparatus 100 recognizes a user's face by using a camera, registers recognized face information or recognizes a user's voice by using a microphone, and registers the recognized voice information. The image display apparatus 100 may also recognize the user's fingerprint, the user's ECG pattern, the PPG pattern, the shape of the back of the hand or a wrist of the user, or a user's iris pattern, and stores the recognized biometric information.

The image display apparatus 100 registers an external device for receiving the registered user health information.

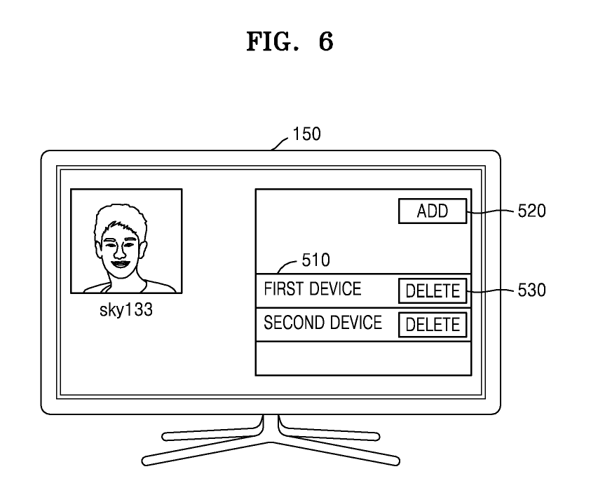

FIG. 6 illustrates a screen through which an external device is registered (or an external device registration screen) according to an exemplary embodiment.

Referring to FIG. 6, the image display apparatus 100 displays an external device registration screen corresponding to a registered user (a first user) on the display unit 150. For example, on the registration screen, user information such as a picture or an ID of the registered user may be displayed and a list 510 of currently registered external devices may be displayed.

As illustrated in FIG. 6, at least one external device (a first device and a second device) may be registered as external devices corresponding to a first user. If health information is received from the external devices (the first device and the second device) registered corresponding to the first user, the image display apparatus 100 recognizes the received health information as health information of the first user.

As illustrated in FIG. 6, the user may select an add button 520 displayed on the registration screen to additionally register an external device.

For example, if the user selects the add button 520, the image display apparatus 100 automatically searches for a health information measuring device near the image display apparatus 100 by using short-range communication. Furthermore, the image display apparatus 100 may display found health information measuring devices as a list on the display unit 150, and the user selects one of the found health information measuring devices to register the selected health information measuring device as an external device corresponding to the user.

The user may also select a delete button 530 to delete a registered external device from the list 510.

Referring back to FIG. 4, the image display apparatus 100 periodically receives health information from the external device 30 in operation S320. The external device 30 may include various devices capable of measuring health information. For example, the external device 30 may include a remote control device including a biometric information measurement sensor to measure biometric information, a mat or device capable of measuring a body weight or body components of a user, glasses capable of measuring brain waves of the user, a heartbeat measuring device for measuring a user's heartrate, a pulse measuring device for measuring a user's pulse, a tonometer for measuring a user's blood pressure, a blood sugar level measuring device for measuring a user's blood sugar level, a pedometer or calories measuring device for measuring a user's activity, a sleep monitoring device capable of measuring biometric information during sleep, a wearable smart device or fitness band, and so forth.

The external device 30 may also be a device registered corresponding to a user as described above. Once an external device for measuring user health information is registered, the image display apparatus 100 may periodically receive the health information from the registered external device.

For example, as illustrated in FIG. 6, once the external devices (the first device and the second device) corresponding to the first user are registered, the image display apparatus 100 may periodically receive health information of the first user from the registered external devices (the first device and the second device).

The image display apparatus 100 may receive the health information from the external devices through at least one of the network interface unit 120 and the external device interface unit 130 illustrated in FIG. 2 and the communication unit 250 illustrated in FIG. 3.

The image display apparatus 100 may receive health information from the external devices by using short-range communication such as Bluetooth®, RFID, IrDA, UWB, ZigBee, NFC, or the like or by using the Internet such as a WLAN, Wi-Fi, WiBro, Wimax, HSDPA, or the like.

The image display apparatus 100 measures ambient environment information through the sensor unit 260. For example, the image display apparatus 100 may periodically measure temperature information, humidity information, cleanliness level information, VOC density information, and air pollution level information around the image display apparatus 100, and store the measured ambient environment information.

Referring back to FIG. 4, the image display apparatus 100 matches and stores the received health information with the user information in operation S330. The image display apparatus 100 recognizes which user among users registered in the image display apparatus 100 the received health information corresponds to, and matches and stores the received health information with its corresponding user information. For example, as mentioned above, the image display apparatus 100 may match and store the health information received from the external devices (the first device and the second device) registered corresponding to the first user with registered user information of the first user (or first user information).

The image display apparatus 100 compares the received health information with previously-stored user health information. If health information patterns of the received health information and the previously-stored user health information coincide within a predetermined range, the image display apparatus 100 matches and stores the received health information with its corresponding user information. For example, if an ECG pattern of the first user coincides with a received ECG within a predetermined range, the image display apparatus 100 stores received ECG information as ECG information of the first user.

When the image display apparatus 100 receives the health information, the image display apparatus 100 may receive user ID information or authentication information or the user ID information or authentication information may be input to the image display apparatus 100. The image display apparatus 100 compares the received or input user ID information or authentication information with previously-registered user ID information or authentication information to match and store the received health information with user information corresponding to the coinciding user ID information or authentication information.

For example, the external device may include at least one of a fingerprint sensor, a camera, and a microphone to recognize a user's fingerprint, face, or voice. If the recognized fingerprint, face, or voice coincides with those of the first user registered, the image display apparatus 100 matches and stores the health information received from the external device with the first user information.

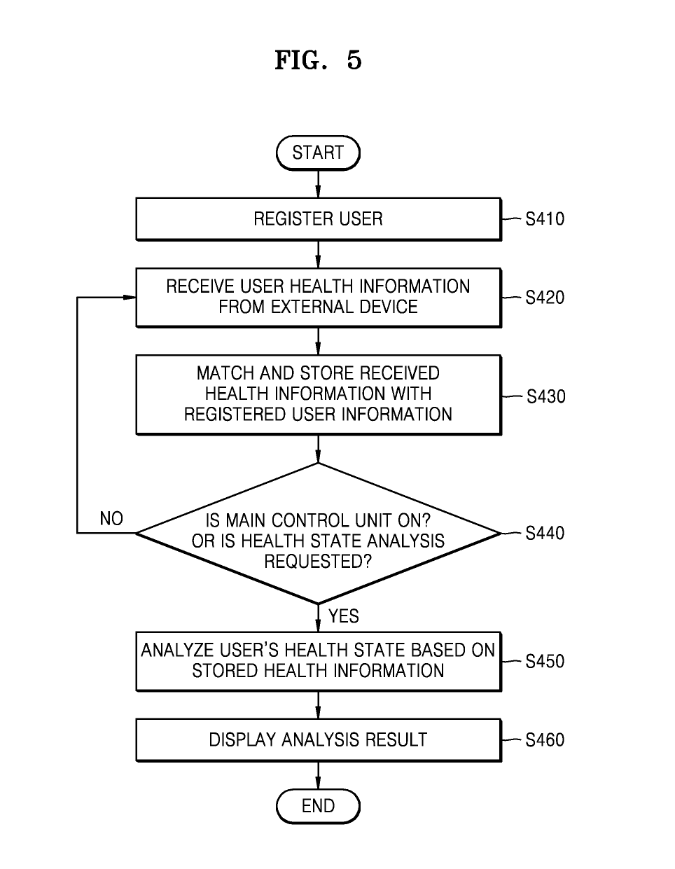

FIG. 5 is a flowchart of an operating method of an image display apparatus 100 according to an exemplary embodiment.

Operations S410 through S430 of FIG. 5 correspond to operations S310 through S330 of FIG. 4 and thus will not be described again.

Referring to FIG. 5, if the user turns on the image display apparatus 100 (for example, if the main controller 140 of FIG. 2 is powered on) or analysis of a user's health state is requested in operation S440, the image display apparatus 100 analyzes stored health information in operation S450. Furthermore, if the user turns on the image display apparatus 100 or analysis of a user's health state is requested, the main control unit 140 stores (e.g., copies or moves) the health information and the ambient environment information stored in the second storing unit 230 in the first storing unit 160.

For example, if the main control unit 140 is powered on or an input for requesting analysis of the user's health state is received, the image display apparatus 100 may analyze the user's health state or mood based on user health information stored in the health information monitoring module 200. In this case, by using ambient environment information measured in the image display apparatus 100, the user's health state or mood may be analyzed.

If the main control unit 140 is powered on or an input for requesting analysis of a registered health state of the first user is received, the image display apparatus 100 may request registered authentication information of the first user. If authentication information input by the user coincides with the registered authentication information of the first user, the image display apparatus 100 analyzes the health state or mood of the first user based on the health information matched and stored with the first user information.

The image display apparatus 100 analyzes the health state or mood of the first user by using the ambient environment information and the health information of the first user, and analyzes recommended environment information based on the ambient environment information and the health information of the first user. The recommended environment information may include recommended temperature, recommended humidity, and recommended cleanliness level of the ambient environment corresponding to the health state of the first user.

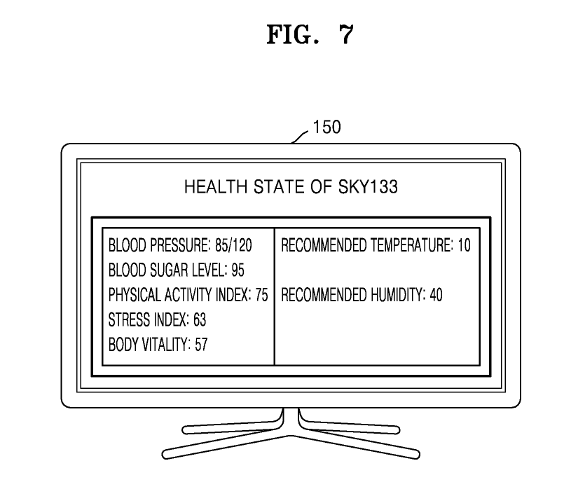

The image display apparatus 100 displays an analysis result on the display unit 150 in operation S460. For example, as illustrated in FIG. 7, the image display apparatus 100 may display blood sugar level information, blood pressure information, brain wave information, ECG/PPG information, GSR information, and activity information of the user, including a body activity index, a stress index, and a body vitality calculated based on the user health information.

The image display apparatus 100 may also display analyzed recommended environment information based on the ambient environment information and the user health information. For example, as illustrated in FIG. 7, the image display apparatus 100 may calculate a recommended temperature, a recommended humidity, and a recommended cleanliness level corresponding to the user's health state and display them on the display unit 150.

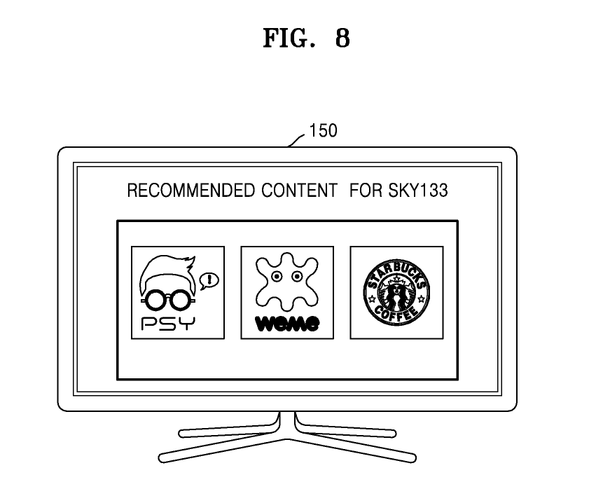

The image display apparatus 100 displays content recommended based on the analyzed user's health state or mood on the display unit 150. For example, as illustrated in FIG. 8, the image display apparatus 100 may display TV programs, movie content, music content, game content, or the like recommended based on the user's health state or mood on the display unit 150.

The image display apparatus 100 also determines whether the user's health state needs medical treatment. In this case, if the image display apparatus 100 determines that the user's health state needs medical treatment, the image display apparatus 100 displays a medical subject or a location of a hospital or drug store, together with a message indicating a need for medical treatment, on the display unit 150.

If the image display apparatus 100 receives an input for requesting user health information from an external device, the image display apparatus 100 transmits stored user health information to the external device. The image display apparatus 100 may request user authentication information to determine whether input user authentication information coincides with registered user authentication information, and transmits the user health information to the external device if the input user authentication information coincides with the registered user authentication information. The image display apparatus 100 may also transmit the user health information to an external device registered corresponding to the user information.

The operating method of the image display apparatus 100 according to the present disclosure can also be implemented through computer-readable code/instructions in/on a medium, e.g., a computer-readable medium, to control at least one processing element to implement any above-described exemplary embodiment. The medium can correspond to any medium/media permitting the storage and/or transmission of the computer-readable code. The computer-readable code can be recorded/transferred on a medium in a variety of ways, with examples of the medium including recording media, such as magnetic storage media (e.g., ROM, floppy disks, hard disks, etc.) and optical recording media (e.g., CD-ROMs, or DVDs), and transmission media such as Internet transmission media. Thus, the medium may be such a defined and measurable structure including or carrying a signal or information, such as a device carrying a bitstream according to one or more exemplary embodiments. The media may also be a distributed network, so that the computer-readable code is stored/transferred and executed in a distributed fashion. Furthermore, the processing element could include a processor or a computer processor, and processing elements may be distributed and/or included in a single device. In this regard, it is understood that one or more of the above-described components may be implemented as software, hardware (e.g., at least one processor, a memory, circuitry, etc.), or a combination of software and hardware.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. An operating method of an image processing apparatus, the operating method comprising:
periodically receiving, from an external device, health information of a user;
matching and storing, in association with the user, the received health information;
analyzing the stored health information; and
outputting a health care service based on a result of the analyzing.

2. The operating method of claim 1, wherein the receiving the health information and the storing the health information are performed in a low-power mode of the image processing apparatus.

3. The operating method of claim 2, wherein the receiving the health information and the storing the health information are performed in the low power mode in which a main controller of the image processing apparatus is powered off.

4. The operating method of claim 1, further comprising:
registering user information of the user,
wherein the registered user information comprises at least one of user Identification (ID) information and user authentication information.

5. The operating method of claim 1, further comprising:
registering, in the image processing apparatus, the external device corresponding to the user,
wherein the periodically receiving the health information comprises periodically receiving the health information of the user from the registered external device.

6. The operating method of claim 1, wherein the health information of the user comprises at least one of biometric information, body weight information, body component information, brain wave information, blood pressure information, blood sugar level information, activity information indicating a user's activity, and sleep monitoring information of the user.

7. The operating method of claim 1, further comprising:
receiving ambient environment information,
wherein the analyzing the stored health information comprises analyzing the stored health information of the user based on the received ambient environment information.

8. The operating method of claim 7, wherein the ambient environment information comprises at least one of a temperature, a humidity, a cleanliness level, and a Volatile Organic Compound (VOC) density.

9. The operating method of claim 1, wherein the outputting the health care service comprises outputting for display recommended content based on the analyzed health information.

10. The operating method of claim 1, wherein the outputting the health care service comprises outputting for display a health state of the user based on a result of the analyzing.

11. An image display apparatus comprising:
a health information monitor configured to receive and store health information of a user;
a main controller configured to analyze the stored health information; and
a display configured to display a result of the analyzing,
wherein the health information monitor comprises:
a communicator configured to periodically receive, from an external device, the health information of the user; and
a storage configured to match and store, in association with the user, the received health information.

12. The image display apparatus of claim 11, wherein the health information monitor further comprises a sub controller configured to control the receiving and the storing of the health information, and the sub controller operates in a low power mode.

13. The image display apparatus of claim 12, wherein the sub controller is configured to receive and store the health information even when the main controller is powered off.

14. The image display apparatus of claim 11, wherein the storage is configured to store user information of the user, which comprises at least one of user Identification (ID) information and user authentication information.

15. The image display apparatus of claim 11, wherein the storage is configured to store external device information corresponding to the user, and the communicator periodically receives the health information of the user from the external device corresponding to the stored external device information.

16. The image display apparatus of claim 11, wherein the health information of the user comprises at least one of biometric information, body weight information, body component information, brain wave information, blood pressure information, blood sugar level information, activity information indicating a user's activity, and sleep monitoring information of the user.

17. The image display apparatus of claim 11, further comprising:
a sensor configured to sense ambient environment information,
wherein the main controller is configured to analyze the stored health information of the user based on the received ambient environment information.

18. The image display apparatus of claim 17, wherein the ambient environment information comprises at least one of a temperature, a humidity, a cleanliness level, and a Volatile Organic Compound (VOC) density.

19. The image display apparatus of claim 11, wherein the display is configured to display recommended content based on the analyzed health information.

20. The image display apparatus of claim 11, wherein the display is configured to output a health state of the user based on a result of the analyzing.

21. A non-transitory computer-readable recording medium having recorded thereon a program for executing the method of claim 1 on a computer.

22. The image display apparatus of claim 13, further comprising:
a main storage,
wherein the main controller is configured to copy or move the received health information, stored in the storage, to the main storage in response to the main controller being powered on, and
wherein the main controller is configured to analyze the health information stored in the main storage.
